SEQUENCE LISTING (13 sequences)

SEQ ID NO: 1 - DNA, 20 bp, Artificial Sequence
  Synthetic
gaggggccggagtattggga

SEQ ID NO: 2 - DNA, 21 bp, Artificial Sequence
  Synthetic
tgtaatccttgccgtcgtagg

SEQ ID NO: 3 - DNA, 16 bp, Artificial Sequence
  Synthetic
ccgctactacaaccag

SEQ ID NO: 4 - DNA, 16 bp, Artificial Sequence
  Synthetic
cggctactacaaccag

SEQ ID NO: 5 - DNA, 19 bp, Artificial Sequence
  Synthetic
ttgggaccgggagacacag

SEQ ID NO: 6 - DNA, 19 bp, Artificial Sequence
  Synthetic
cgatgtaatccttgccgtc

SEQ ID NO: 7 - DNA, 14 bp, Artificial Sequence
  Synthetic
ccgagtgagcctgc

SEQ ID NO: 8 - DNA, 14 bp, Artificial Sequence
  Synthetic
ccgagtgaacctgc

SEQ ID NO: 9 - DNA, 14 bp, Artificial Sequence
  Synthetic
catcgctggtgctc

SEQ ID NO: 10 - DNA, 15 bp, Artificial Sequence
  Synthetic
cattgctggtgctcc

SEQ ID NO: 11 - DNA, 22 bp, Artificial Sequence
  Synthetic
ctcttcatcctcctcttctttc

SEQ ID NO: 12 - DNA, 21 bp, Artificial Sequence
  Synthetic
gaaaacgcagtgattcaactg

SEQ ID NO: 13 - protein, 6 aa, Artificial Sequence
  Synthetic
MGBNFQ

(19) United States
(12) Patent Application Publication
(10) Pub. No.: US 2013/0267613 A1
(43) Pub. Date: Oct. 10, 2013

(54) MOLECULAR-DETERMINANT BASED TYPING OF KIR ALLELES AND KIR-LIGANDS

(71) Applicants: Wing Leung, Memphis, TN (US); Rafijul Bari, Bartlett, TN (US)

(72) Inventors: Wing Leung, Memphis, TN (US); Rafijul Bari, Bartlett, TN (US)

(73) Assignee: St Jude Children's Research Hospital, Memphis, TN (US)

(21) Appl. No.: 13/877,006

(22) PCT Filed: Oct. 5, 2011
(86) PCT No.: PCT/US11/54906

§ 371 Date: Apr. 26, 2013

Related U.S. Application Data
(60) Provisional application No. 61/390,473, filed on Oct. 6, 2010.

(51) Int. Cl. C12Q 1/68

(52) U.S. Cl. CPC C12Q 1/6883
USPC 514/789; 536/24.31; 435/6.11

(57) ABSTRACT

The present invention relates to an assay to perform a molecular determinant-based functional killer immunoglobulin-like receptors (KIR) allele typing and ligand typing. In particular the present invention provides methods, compositions, and kits for a single nucleotide polymorphism (SNP) assay to type various allele groups of KIR2DL1 and KIR ligand with distinct functional properties based on polymorphism at position 245 in KIR2DL1, position 77 in HLA-C, and position 83 in HLA-B and HLA-A. The assays are suitable for use in predicting NK cell activity in health, disease, and transplantation.

### CROSS-REFERENCE TO RELATED APPLICATIONS

This application claims the benefit of U.S. Provisional Application No. 61/390,473, filed on Oct. 6, 2010.

### FIELD OF THE INVENTION

The present invention relates to an assay to perform a molecular determinant-based functional killer immunoglobulin-like receptor (KIR) allele typing and ligand typing. In particular the present invention provides methods, compositions, and kits for a single nucleotide polymorphism (SNP) assay to type various allele groups of KIR2DL1 and KIR ligand with distinct functional properties based on polymorphism at position 245 in KIR2DL1, position 77 in HLA-C, and position 83 in HLA-B and HLA-A. The assays are suitable for use in predicting NK cell activity in health, disease, and transplantation.

### BACKGROUND OF THE INVENTION

Natural killer (NK) and T cells play central and complementary functions in immunity against transformed or virus-infected cells (Vely et al. 2001). NK cells distinguish between healthy and abnormal cells by using a repertoire of cell surface receptors that control their activation, proliferation, and effector functions (Lanier et al. 2005). A hallmark of human NK cells is the expression of HLA class I-specific killer-cell immunoglobulin-like receptors (KIR) (Uhrberg et al. 2005).

KIRS are not only variably expressed on the level of single NK cells but they are highly polymorphic and polygenic, such that the gene content of the KIR cluster varies from individual to individual (Uhrberg et al. 2005). There are 15 KIR genes (plus two pseudogenes) known to date, 11 encoding receptors with two immunoglobulin domains (KIR2D genes) and 4 with three domains (KIR3D). The KIR family is further divided into inhibitory and stimulatory KIR. The inhibitory KIR genes are characterized by long cytoplasmic tails (indicated in their name by “L” for long) featuring immunoreceptor tyrosine-based inhibitory motifs (ITIM). In contrast, stimulatory KIR have short cytoplasmic tails (indicated in their name by “S” for short) lacking ITIM, but have a charged amino acid in the transmembrane region that provides a docking site for the activating molecule (Uhrberg et al. 2005).

Many human diseases are reported to be associated with differences in KIR gene content, including autoimmune diseases, inflammatory disorders, infectious diseases, immunodeficiency, cancer, and reproductive disorders (Kulkarni et al. 2008). The relation between these diseases and functional heterogeneity among the alleles of KIR is not yet known due to the lack of rapid methods for high throughput typing of different functional groups of KIR alleles.

Use of KIR genotyping for transplants, among other medical procedures is disclosed in:

Lebedeva et al., “Comprehensive Approach to High-Resolution KIR Typing” Hum Immun., 68(9): 789-96 (2007); Gonzalez et al., “Killer Cell Immunoglobulin-Like Receptor Allele Discrimination by High-Resolution Melting” Hum Immun., 70(10):858-63 (2009); Yun et al., “A Novel Method for KIR-Ligand Typing by Pyrosequencing To Predict NK Cell Alloreactivity” Blood (ASH Annual Meeting Abstracts) 106:Abstract 1407 (2005) (Also see Yun et al., “A Novel Method for KIR-Ligand Typing by Pyrosequencing To Predict NK Cell Alloreactivity” Clin Immunol. 123(3):272-280 (2007).); Leung et al., “Comparison of Killer Ig-Like Receptor Genotyping and Phenotyping for Selection of Allogeneic Blood Stem Cell Donors” J Immun. 174:6540-6545 (2005); Dinauer et al., “Primers, Methods and Kits For Detecting Killer-Cell Immunoglobulin-Like Receptor Alleles” U.S. Patent Application Publication No. US 2008/0280289 (See also International Application Publication No. WO 2005/046459 selected parts; and KIR Genotyping Product Brochure 2004.); Chen et al., “Natural Killer Immunoglobulin-Like Receptor (KIR) Assay” International Application Publication No. WO 2009/051672. Also see PCT/US2008/011671; Trachtenberg et al., “Methods and Compositions For KIR Genotyping” U.S. Patent Application Publication No. US 2008/0213787 (Also see International Application Publication No. WO 2007/041067.); Houtchens et al., “High-Throughput Killer Cell Immunoglobulin-Like Receptor Genotyping By MALDI-TOF Mass Spectrometry With Discovery of Novel Alleles” Immunogenetics. 59(7):525-37 (2007); Gómez-Lozano et al., “Genotyping of Human Killer-Cell Immunoglobulin-Like Receptor Genes by Polymerase Chain Reaction with Sequence-Specific Primers: An Update” Tissue Antigens 59(3):184-193 (2002); and Shilling et al., “Allelic Polymorphism Synergizes with Variable Gene Content to Individualized Human KIR Genotype” J Immunol. 168:2307-2315 (2002). Each of which are hereby incorporated by reference in their entirety.

Moreover some KIR genotyping kits available include, limo-Train, “KIR-Ready Gene” Product Brochure 9/2005; Miltenyi Biotec, “KIR Typing Kit” Product Brochure 2009; Invitrogen, “KIR Genotyping SSP Kit” Product Brochure 11/2006; and Tepnel Lifecodes, “KIR Genotyping” Product Brochure 6/2005. Each of which are hereby incorporated by reference in their entirety.

Thus, because disease susceptibility has been associated with various KIR ligand constellations and allele typing plays an important role in the success of transplants what is needed are novel methods, compositions and kits for detecting the molecular determinants of both KIRs and their ligands.

### SUMMARY OF THE INVENTION

The present invention relates to an assay to perform a molecular determinant-based functional killer immunoglobulin-like receptor (KIR) allele typing and ligand typing. In particular the present invention provides methods, compositions, and kits for a single nucleotide polymorphism (SNP) assay to type various allele groups of KIR2DL1 and KIR ligand with distinct functional properties based on polymorphism at position 245 in KIR2DL1, position 77 in HLA-C, and position 83 in HLA-B and HLA-A. The assays are suitable for use in predicting NK cell activity in health, disease, and transplantation.

Killer cell immunoglobulin-like receptors (KIR) regulate NK cell function. KIRs and their HLA ligands are highly polymorphic in nature with substantial allelic polymorphism. At present, there is a lack of expedient method for KIR- and HLA-allele typing with relevant functional information. In one embodiment, the present invention contemplates a single nucleotide polymorphism (SNP) assay to type various allele groups of KIR2DL1 with distinct functional properties based on polymorphism at position 245. In one embodiment, the present invention contemplates a SNP assay to type different KIR-ligands based on polymorphism at position 77 in HLA-C and position 83 in HLA-B and -A. It is believed that the present SNP assays for KIR and KIR-ligand typing are much cheaper and faster as compared to existing high resolution typing. Importantly, the high throughput methods provide results that are informative in predicting NK cell activity in health, disease, and transplantation.

While it is not the intention that the present invention be limited to KIR allele typing, in one embodiment, the present invention contemplates a probe comprising the nucleic acid sequence selected from the group consisting of a nucleic acid sequence of SEQ. ID. NO: 9, a nucleic acid sequence of SEQ. ID. NO: 10, a nucleic acid sequence with greater than 98 percent homology of SEQ. ID. NO: 9, a nucleic acid sequence with greater than 98 percent homology of SEQ. ID. NO: 10, a nucleic acid sequence with greater than 95 percent homology of SEQ. ID. NO: 9, a nucleic acid sequence with greater than 95 percent homology of SEQ. ID. NO: 10, a nucleic acid sequence with greater than 90 percent homology of SEQ. ID. NO: 9, a nucleic acid sequence with greater than 90 percent homology of SEQ. ID. NO: 10, wherein said probe is capable of distinguishing between the presence and absence of nucleic acid sequences coding for arginine and cysteine at position 245 of KIR2DL1.

In another embodiment, the present invention provides a kit, comprising: a) providing: i. a probe from described above and ii. instructions for use.

In another embodiment, the present invention provides a method, comprising: a) providing: i. a sample from a subject, wherein said sample comprises nucleic acid encoding KIRs; ii. a plurality of primers wherein said primers can amplify all of the alleles of KIR2DL1; iii. a plurality of probes wherein said probes can recognize the presence of nucleic acid sequences coding for arginine and cysteine at position 245; and b) contacting said sample with said primers for a sufficient amount of time to amplify all of the alleles of KIR2DL1; and c) determining the presence and absence of nucleic acid sequences coding for arginine and cysteine with said plurality of probes. In some embodiments the method further comprises treating the subject with a first therapy depending on the presence of coding sequence for arginine. In still other embodiments the method further comprises treating the subject with a second therapy depending on the presence of coding sequence for cysteine.

In another embodiment, the present invention provides a kit, comprising: a) providing: i. a plurality of primers and a plurality of probes for KIR typing wherein said typing includes distinguishing between the presence and absence of nucleic acid sequences coding for arginine and cysteine at position 245 of KIR2DL1; and ii. instructions for use.

In still another embodiment, the present invention provides a probe comprising the nucleic acid sequence selected from the group consisting of a nucleic acid sequence of SEQ. ID. NO: 7, a nucleic acid sequence of SEQ. ID. NO: 8, a nucleic acid sequence with greater than 98 percent homology of SEQ. ID. NO: 7, a nucleic acid sequence with greater than 98 percent homology of SEQ. ID. NO: 8, a nucleic acid sequence with greater than 95 percent homology of SEQ. ID. NO: 7, a nucleic acid sequence with greater than 95 percent homology of SEQ. ID. NO: 8, a nucleic acid sequence with greater than 90 percent homology of SEQ. ID. NO: 7, a nucleic acid sequence with greater than 90 percent homology of SEQ. ID. NO: 8, wherein said probe is capable of distinguishing between the presence and absence of nucleic acid sequences coding for serine and asparagine at position 77 of HLA-C1/C2.

In some embodiments, the present invention provides a kit, comprising: a) providing: i. a probe as described above; and ii. instructions for use.

In another embodiment, the present invention provides a method, comprising: a) providing: i. a sample from a subject, wherein said sample comprises nucleic acid encoding MR ligands; ii. a plurality of primers wherein said primers can amplify all of the alleles of HLA-C1/C2; iii. a plurality of probes wherein said probes can recognize the presence of nucleic acid coding for a serine and asparagine at position 77 of HLA-C1/C2; and b) contacting said sample with said primers for a sufficient amount of time to amplify all of the alleles of HLA-C1/C2; and c) determining the presence and absence of nucleic acid sequences coding for serine and asparagine with said plurality of probes. In some embodiments the method further comprises treating the subject with a first therapy depending on the presence of coding sequence for serine. In some embodiments the method further comprises treating the subject with a second therapy depending on the presence of coding sequence for asparagine.

In yet another embodiment, the present invention provides a kit, comprising: a) providing: i. a plurality of primers and a plurality of probes for KIR ligand typing wherein said typing includes distinguishing between the presence and absence of nucleic acid sequences coding for serine and asparagine at position 77 of HLA-C1/C2; and ii. instructions for use.

In still another embodiment, the present invention contemplates a probe comprising the nucleic acid sequence selected from the group consisting of a nucleic acid sequence of SEQ. ID. NO: 3, a nucleic acid sequence of SEQ. ID. NO: 4, a nucleic acid sequence with greater than 98 percent homology of SEQ. ID. NO: 3, a nucleic acid sequence with greater than 98 percent homology of SEQ. ID. NO: 4, a nucleic acid sequence with greater than 95 percent homology of SEQ. ID. NO: 3, a nucleic acid sequence with greater than 95 percent homology of SEQ. ID. NO: 4, a nucleic acid sequence with greater than 90 percent homology of SEQ. ID. NO: 3, a nucleic acid sequence with greater than 90 percent homology of SEQ. ID. NO: 4, wherein said probe is capable of distinguishing between the presence and absence of nucleic acid sequences coding for arginine in HLA-Bw4 and HLA-A, and glycine in HLA-Bw6 at position 83.

In yet another embodiment, the present invention provides a kit, comprising: a) providing: i. a probe from above; and ii. instructions for use.

In another embodiment, the present invention contemplates a method, comprising: a) providing: i. a sample from a subject, wherein said sample comprises nucleic acid encoding KIR ligands HLA-B and -A; ii. a plurality of primers wherein said primers can amplify all of the alleles of HLA-B/A; iii. a plurality of probes wherein said probes can recognize the presence of nucleic acid coding for a arginine in HLA-Bw4 and HLA-A and a glycine in HLA-Bw6 at position 83; and b) contacting said sample with said primers for a sufficient amount of time to amplify all of the alleles of HLA-B/A; and c) determining the presence and absence of nucleic acid sequences coding for arginine and glycine with said plurality of probes. In some embodiments the method further comprises treating the subject with a first therapy depending on the presence of coding sequence for arginine. In some embodiments the method further comprises treating the subject with a second therapy depending on the presence of coding sequence for glycine.

In yet other embodiments the present invention provides a kit, comprising: a) providing: i. a plurality of primers and a plurality of probes for KIR ligands typing wherein said typing includes distinguishing between the presence and absence of nucleic acid sequences coding for arginine in HLA-Bw4 and HLA-A, and glycine in HLA-Bw6 at position 83; and ii. instructions for use.

While specific forward and reverse primers and probes are presented within as sequences the specific sequences are not meant to be limiting and include complementary and reverse complimentary (sense and anti-sense strings, comparable identities (with similarity and identity of sequences of about 70-100%, preferably about 80-100%, more preferably about 85-100%, even more preferably about 90-100%, and most preferably about 95-100%), homologs, mimetics, portions/fragments thereof, 5′-3′ or 3′-5′ order) sequences as known by one of skill in the art. Furthermore, descriptions of embodiments presented are not meant to be limiting and include all equivalent, comparable technologies, reagents, sources, diluents, uses etc. as known by one skilled in the art. Moreover, KIR allele typing and ligand typing is contemplated to be applied broadly to disease detection/diagnosis, management, and therapeutic/non-therapeutic treatment along with applications for other polymorphic loci within the genome of mammals. For example only, and not meant to be limiting, more particularly humans but also includes dogs, cats, horses, rat, mice, hamster, bovine, sheep, goat, pigs, among other non-human mammals.

### BRIEF DESCRIPTION OF THE FIGURES

FIG. 1 shows molecular determinant based KIR typing predicts NK cell activity. CD56+ cells were isolated from donors PBMCs using Automacs (Miltenyi Biotech). Isolated CD56+ cells were mixed with target 721.221-Cw7 or 721.221-Cw6. After CD107 labeling procedure, cells were stained with different KIR mAb. KIR2DL1+ NK cell subsets were gated and surface expression of CD107 was detected using flow cytometry. (A). Surface expression of CD107 on NK cells without any target cells; (B). Degranulation of KIR2DL1 R²⁴⁵/R²⁴⁵ positive NK cell subset against 721.221 cell expressing KIR2DL1 non-ligand HLA-Cw7; (C). Degranulation of KIR2DL1 R²⁴⁵/R²⁴⁵ positive NK cell subset against 721.221 cell expressing KIR2DL1 ligand HLA-Cw6; (D). Degranulation of KIR2DL1 C²⁴⁵/C²⁴⁵ positive NK cell subset against 721.221 cell expressing KIR2DL1 non-ligand HLA-Cw7; (E). Degranulation of KIR2DL1 C²⁴⁵/C²⁴⁵ positive NK cell subset against 721.221 cell expressing KIR2DL1 ligand HLA-Cw6.

FIG. 2 shows KIR-ligand group typing predicts NK cell activity, Donor PBMCs pre-labeled with CD45 and mixed with K562. Single KIR+ NK cell subsets were gated using mAb against KIRs. The sequences of panels demonstrate the gating strategy of single KIR+ (first two panels) and degranulation of KIR2DL1+ (lowest panel) NK cell subset.

FIG. 3 shows donor PBMCs pre-labeled with CD45 and mixed with K562. Single KIR+ NK cell subsets were gated as described in FIG. 2. (A). Degranulation of KIR2DL2/2DL3+ subset; (B). Degranulation of KIR3DL1+ subset; and (C). Reactivity of 5 donors KIR2DL1+ and KIR2DL2/2DL3+ NK cell subsets toward K562 cells.

FIG. 4 shows reactivity of NK cell subsets can be predicted based on KIR ligand group typing. KIR-ligand typing of donor cells were done using SNP assay and then followed by prediction of NK cell licensing. Single KIR+ NK cell subsets were gated using mAb against KIRs, and NK cells degranulation were measure using CD 107 marker. Reactivity of NK cell subsets was determined toward target cells ectopically expressing their ligands. Shown are reactivity of (A). Educated (left panel, p value<0.05) and uneducated (right panel) KIR2DL1+ NK cell subset toward 721.221 cells expressing their ligand Cw6 or non-ligand Cw7; and (B). Educated (left panel, p value<0.05) and uneducated (right panel) KIR2DL2/2DL3+ NK cell subset toward 721.221 expressing their non-ligand Cw6 or ligand Cw7.

FIG. 5 shows functional group typing of KIR2DL1. DNA from donor PBMCs was extracted. Probes were designed for different functional groups of KIR2DL1 alleles based on single nucleotide polymorphism. Primers were designed that can specifically amplify all the alleles of KIR2DL1. KIR2DL1 allele typing was done using HT7900 from Applied Biosystems. KIR2DL1 C²⁴⁵/C²⁴⁵ is shown in blue dots; KIR2DL1 R²⁴⁵/C²⁴⁵ is shown in green dots; KIR2DL1 R²⁴⁵/R²⁴⁵ is shown in red dots; Negative control is shown as a black box; and undetermined is shown as a black X.

FIG. 6 shows functional group typing of KIR ligands. DNA from donor PBMCs was extracted. Probes were design to type KIR-ligands based on single nucleotide mismatch. A) Typing of KIR2DL1 and KIR2DL2/2DL3 ligands HLA-C, (HLA-C2/HLA-C2 is shown in blue dots; HLA-C1/HLA-C2 is shown in green dots; HLA-C1/HLA-C1 is shown in red dots; and Negative Control is shown as a black box; B) Typing of KIR3DL1 ligand HLA-Bw4 (HLA-Bw4/HLA-Bw4 is shown in blue dots; HLA-Bw4/HLA-Bw6 is shown in green dots; HLA-Bw6/HLA-Bw6 is shown in red dots; and Negative Control is shown as a black box).

### DEFINITIONS

To facilitate the understanding of this invention a number of terms (set off in quotation marks in this Definitions section) are defined below. Terms defined herein (unless otherwise specified) have meanings as commonly understood by a person of ordinary skill in the areas relevant to the present invention.

Unless otherwise indicated, all numbers expressing quantities of ingredients, properties such as molecular weights, reaction conditions, and so forth as used in the specification and claims are to be understood as being modified in all instances by the term “about.” Accordingly, unless indicated to the contrary, the numerical parameters in the specification and claims are approximations that may vary depending upon the desired properties sought to be obtained by the present invention. At the very least, and without limiting the application of the doctrine of equivalents to the scope of the claims, each numerical parameter should at least be construed in light of the number of reported significant digits and by applying ordinary rounding techniques. Notwithstanding that the numerical ranges and parameters describing the broad scope of the invention are approximations, the numerical values in the specific examples are reported as precisely as possible. Any numerical value, however, inherently contains standard deviations that necessarily result from the errors found in the numerical value's testing measurements.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to delivery systems comprising two or more separate containers that each contains a subportion of the total kit components. The containers may be delivered to the intended recipient together or separately. For example, a first container may contain an enzyme for use in an assay, while a second container contains oligonucleotides. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

As used herein, the term “subject” or “patient” refers to any organism to which compositions in accordance with the invention may be administered, e.g., for experimental, diagnostic, prophylactic, and/or therapeutic purposes. Typical subjects include animals (e.g., mammals such as mice, rats, rabbits, non-human primates, and humans; insects; worms; etc.).

As used herein, the term “single nucleotide polymorphism” or “SNP”, refers to any position along a nucleotide sequence that has one or more variant nucleotides. Single nucleotide polymorphisms (SNPs) are the most common form of DNA sequence variation found in the human genome and are generally defined as a difference from the baseline reference DNA sequence which has been produced as part of the Human Genome Project or as a difference found between a subset of individuals drawn from the population at large. SNPs occur at an average rate of approximately 1 SNP/1000 base pairs when comparing any two randomly chosen human chromosomes. Extremely rare SNPs can be identified which may be restricted to a specific individual or family, or conversely can be found to be extremely common in the general population (present in many unrelated individuals). SNPs can arise due to errors in DNA replication (i.e., spontaneously) or due to mutagenic agents (i.e., from a specific DNA damaging material) and can be transmitted during reproduction of the organism to subsequent generations of individuals.

As used herein, the term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 KB or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “transgene” refers to a heterologous gene that is integrated into the genome of an organism (e.g., a non-human animal) and that is transmitted to progeny of the organism during sexual reproduction.

As used herein, the terms “complementary” or “complementarity” are used in reference to polynucleotides (i.e., a sequence of nucleotides) related by the base-pairing rules. For example, for the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

As used herein, the term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

As used herein, the term “polymerase chain reaction” (“PCR”) refers to the method of K. B. Mullis U.S. Pat. Nos. 4,683,195, 4,683,202, and 4,965,188, hereby incorporated by reference, that describe a method for increasing the concentration of a segment of a target sequence in a DNA mixture without cloning or purification. Because the desired amplified segments of the target sequence become the predominant sequences (in terms of concentration) in the mixture, they are said to be “PCR amplified.” Similarly, the term “modified PCR” as used herein refers to amplification methods in which a RNA sequence is amplified from a DNA template in the presence of RNA polymerase or in which a DNA sequence is amplified from an RNA template the presence of reverse transcriptase.

As used herein the term “stringency” is used in reference to the conditions of temperature, ionic strength, and the presence of other compounds such as organic solvents, under which nucleic acid hybridizations are conducted. “Stringency” typically occurs in a range from about Tₘ to about 20° C. to 25° C. below Tₘ. A “stringent hybridization” can be used to identify or detect identical polynucleotide sequences or to identify or detect similar or related polynucleotide sequences. For example, when fragments are employed in hybridization reactions under stringent conditions the hybridization of fragments which, contain unique sequences (i.e., regions which are either non-homologous to or which contain less than about 50% homology or complementarity) are favored. Alternatively, when conditions of “weak” or “low” stringency are used hybridization may occur with nucleic acids that are derived from organisms that are genetically diverse (i.e., for example, the frequency of complementary sequences is usually low between such organisms).

“Low stringency conditions” comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.1% SDS, 5×Denhardt's reagent {50×Denhardt's contains per 500 ml: 5 g Ficoll (Type 400, Pharmacia), 5 g BSA (Fraction V; Sigma)} and 100 μg/ml denatured salmon sperm DNA followed by washing in a solution comprising 5×SSPE, 0.1% SDS at 42° C. when a probe of about 500 nucleotides in length. is employed. Numerous equivalent conditions may also be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol), as well as components of the hybridization solution may be varied to generate conditions of low stringency hybridization different from, but equivalent to, the above listed conditions. In addition, conditions which promote hybridization under conditions of high stringency (e.g., increasing the temperature of the hybridization and/or wash steps, the use of formamide in the hybridization solution, etc.) may also be used.

“Medium stringency conditions” when used in reference to nucleic acid hybridization comprise conditions equivalent to binding or hybridization at 42.degree. C. in a solution consisting of 5.times.SSPE (43.8 g/l NaCl, 6.9 g/l NaH.sub.2PO.sub.4.H.sub.2O and 1.85 g/l EDTA, pH adjusted to 7.4 with NaOH), 0.5% SDS, 5.times. Denhardt's reagent and 100.mu.g/ml denatured salmon sperm DNA followed by washing in a solution comprising 1.0.times.SSPE, 1.0% SDS at 42.degree. C. when a probe of about 500 nucleotides in length is employed.

“High stringency conditions” when used in reference to nucleic acid hybridization comprise conditions equivalent to binding or hybridization at 42 degrees C. in a solution consisting of 5.times.SSPE (43.8 g/l NaCl, 6.9 g/l NaH.sub.2PO.sub.4.H.sub.2O and 1.85 g/l EDTA, pH adjusted to 7.4 with NaOH), 0.5% SDS, 5.times. Denhardt's reagent and 100.mu.g/ml denatured salmon sperm DNA followed by washing in a solution comprising 0.1.times.SSPE, 1.0% SDS at 42.degrees. C. when a probe of about 500 nucleotides in length is employed.

As used herein, the term “hybridization” is used in reference to the pairing of complementary nucleic acids using any process by which a strand of nucleic acid joins with a complementary strand through base pairing to form a hybridization complex. Hybridization and the strength of hybridization (i.e., the strength of the association between the nucleic acids) is impacted by such factors as the degree of complementarity between the nucleic acids, stringency of the conditions involved, the Tₘ of the formed hybrid, and the G:C ratio within the nucleic acids.

As used herein the term “hybridization complex” refers to a complex formed between two nucleic acid sequences by virtue of the formation of hydrogen bounds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., C₀ t or R₀ t analysis) or between one nucleic acid sequence present in solution and another nucleic acid sequence immobilized to a solid support (e.g., a nylon membrane or a nitrocellulose filter as employed in Southern and Northern blotting, dot blotting or a glass slide as employed in in situ hybridization, including FISH (fluorescent in situ hybridization)).

As used herein, the term “Tₘ” is used in reference to the “melting temperature.” The melting temperature is the temperature at which a population of double-stranded nucleic acid molecules becomes half dissociated into single strands. As indicated by standard references, a simple estimate of the Tₘ value may be calculated by the equation: Tₘ=81.5+0.41 (% G+C), when a nucleic acid is in aqueous solution at 1M NaCl. Anderson et al., “Quantitative Filter Hybridization” In: Nucleic Acid Hybridization (1985). More sophisticated computations take structural, as well as sequence characteristics, into account for the calculation of Tₘ.

As used herein, the term “amplifiable nucleic acid” is used in reference to nucleic acids which may be amplified by any amplification method. It is contemplated that “amplifiable nucleic acid” will usually comprise “sample template.”

As used herein, the term “sample template” refers to nucleic acid originating from a sample, which is analyzed for the presence of a target sequence of interest. In contrast, “background template” is used in reference to nucleic acid other than sample template, which may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxy-ribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers; to an oligonucleotide (i.e., a sequence of nucleotides), whether occurring naturally as in a purified restriction digest or produced synthetically, recombinantly or by PCR amplification, which is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the terms “restriction endonucleases” and “restriction enzymes” refer to bacterial enzymes, each of which cut double-stranded DNA at or near a specific nucleotide sequence.

DNA molecules are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements which direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the term “nucleic acid sequence” refers to an oligonucleotide, a nucleotide or a polynucleotide, and fragments or portions thereof, and vice versus, and to DNA or RNA of genomic or synthetic origin, which may be single or double-stranded, and represent the sense or antisense strand. Similarly, “amino acid sequence” as used herein refers to peptide or protein sequence.

As used herein, the term “antisense” when used in reference to DNA refers to a sequence that is complementary to a sense strand of a DNA duplex. A “sense strand” of a DNA duplex refers to a strand in a DNA duplex that is transcribed by a cell in its natural state into a “sense mRNA.” Thus an “antisense” sequence is a sequence having the same sequence as the non-coding strand in a DNA duplex.

As used herein, the term “amplification reagents” refers to those reagents (deoxyribonucleotide triphosphates, buffer, etc.), needed for amplification except for primers, nucleic acid template, and the amplification enzyme. Typically, amplification reagents along with other reaction components are placed and contained in a reaction vessel (test tube, microwell, etc.).

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the term “vector” refers to a system used to transfer nucleic acid sequences from one cell to another or from one organism to another including but not limited to any means of delivering a composition comprising a nucleic acid sequence to a cell or tissue. For example, vectors include but are not limited to plasmids (e.g., pcDNA3.1), artificial chromosomes, bacteria, fungi, and viruses (e.g., retroviral, adenoviral, adeno-associated viral, and other nucleic acid-based delivery systems etc.).

As used herein, the terms “expression vector,” “expression construct,” “expression cassette” and “plasmid,” refer to a recombinant nucleic acid molecule containing a desired coding sequence and appropriate nucleic acid sequences necessary for the expression of the operably linked coding sequence in a particular host organism. The sequences may be either double or single-stranded. Nucleic acid sequences necessary for expression in prokaryotes usually include a promoter, an operator (optional), and a ribosome-binding site, often along with other sequences. Eukaryotic cells are known to utilize promoters, enhancers, and termination and polyadenylation signals.

As used herein, the terms “in operable combination,” “in operable order,” and “operably linked” refer to the linkage of nucleic acid sequences in such a manner that a nucleic acid molecule capable of directing the transcription of a given gene and/or the synthesis of a desired protein molecule is produced. The terms also refer to the linkage of amino acid sequences in such a manner so that a functional protein is produced.

As used herein, the term “transfection” refers to the introduction of foreign nucleic acid (e.g., DNA) into cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, biolistics (i.e., particle bombardment), and the like.

As used herein, the term “antibody” refers to immunoglobulin evoked in animals by an immunogen (antigen). It is desired that the antibody demonstrate specificity to epitopes contained in the immunogen. The term “polyclonal antibody” refers to immunoglobulin produced from more than a single clone of plasma cells; in contrast “monoclonal antibody” refers to immunoglobulin produced from a single clone of plasma cells. Antibody encompasses, but is not limited to recombinantly prepared, and modified antibodies and antigen-binding fragments thereof, such as chimeric antibodies, humanized antibodies, multifunctional antibodies, bispecific or oligo-specific antibodies, single-stranded antibodies and F(ab) or F(ab).sub.2 fragments.

As used herein, the terms “specific binding” and “specifically binding” when used in reference to the interaction of an antibody and a protein or peptide means that the interaction is dependent upon the presence of a particular structure (i.e., for example, an antigenic determinant or epitope) on a protein; in other words an antibody is recognizing and binding to a specific protein structure rather than to proteins in general. For example, if an antibody is specific for epitope “A”, the presence of a protein containing epitope A (or free, unlabelled A) in a reaction containing labeled “A” and the antibody will reduce the amount of labeled A bound to the antibody.

As used herein, the terms “FACS,” “Flow cytometry,” and “Fluorescent Activated Cell Sorter” are used interchangeably and refer broadly to a system for measuring and analyzing the signals that result from particles as they flow in a liquid stream through a beam of light. For example only, and not meant to be limiting human peripheral blood mononuclear cells (PBMC) carry surface markers that can be fluorescently stained either directly or indirectly using antibodies labeled with fluorescent dyes. As the stained cells pass through the beam of light they generate a signal that is captured and displayed on a screen. The signal includes forward scatter (light refracted based on size), side scatter (light deflected based on granularity), along with fluorescence generated from the dye(s) (See, Givan, A. L., “Flow Cytometry First Principles, 2^{nd} Edition, 2001 herein incorporated by reference).

As used herein, the term “detection assay” refers to an assay for detecting the presence or absence of variant nucleic acid sequences (e.g., subtypes, polymorphism or mutations) in a given allele or nucleic acid (e.g., KIR allele typing).

As used herein, the terms “functional licensing,” “licensing,” and “education” are used interchangeably and refer to the process of NK cells being able to distinguish or recognize self from non-self. More particularly, for example only and not meant to be limiting, NK cells recognize the difference between self and non-self by the presence or absence of surface HLA molecules. NK cells that carry a receptor for self HLA ligand are “licensed” and ready to kill target cells with missing HLA ligand.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used herein, the term “sample” is used in its broadest sense and includes environmental and biological samples. Environmental samples include material from the environment such as soil and water. Biological samples may be animal, including, human, fluid (e.g., blood, plasma and serum), solid (e.g., stool), tissue, liquid foods (e.g., milk), and solid foods (e.g., vegetables). For example, a pulmonary sample may be collected by bronchoalveolar lavage (BAL), which comprises fluid and cells derived from lung tissues. A biological sample may comprise a cell, tissue extract, body fluid, chromosomes or extrachromosomal elements isolated from a cell, genomic DNA (in solution or bound to a solid support such as for Southern blot analysis), RNA (in solution or bound to a solid support such as for Northern blot analysis), cDNA (in solution or bound to a solid support) and the like.

As used herein, the term “therapy” is used in its broadest sense and includes treatment for autoimmune diseases, transplant related disorders, inflammatory disorders, infectious diseases, immunodeficiency, cancer, and reproductive disorders. Specific treatments will depend on the condition being treated and the subjects medical requirements. It is not intended that the present invention be limited to therapy or treatment only where the disease is cured. It is sufficient in some embodiments that one or more symptom of a disease is reduced by such treatment, e.g. inflammation is reduced, fever is reduced, infection is reduced, cancer growth is inhibited, etc. It is sufficient in other embodiments, that one or more condition of the patient is improved by the treatment, e.g. immune function is improved.

### DETAILED DESCRIPTION OF THE INVENTION

Natural killer (NK) cells are a member of the innate immune system and they are important for infection control (Lodoen et al. 2006; Cerwenka et al. 2001), cancer surveillance (Caligiuri, M A 2008; Waldhauer et al. 2008; Kim et al. 2007) and successful pregnancy (Santoni et al. 2008; Moffett-King, A. 2002). NK cell functions are regulated by various activating and inhibitory receptors present on the cell surface (Lather, LL 2008). The major class of inhibitory receptors is killer immunoglobulin-like receptors (KIRs) (Long et al. 1996; Parham, P. 2008), which are highly polymorphic in nature (Robinson et al. 2007). Allelic polymorphism generates functional heterogeneity among the alleles of the same KIR gene.

For example, it was previously shown that KIR2DL1 alleles having arginine at amino acid position 245 have higher inhibitory activity and more durable surface expression upon identical ligand engagement when compared to alleles that have cysteine at the same position (Bari et al. 2009). Different alleles of KIR3DL1 are also reported to have different inhibitory capacity and level of steady-state cell-surface expression (Yawata et al. 2006). Some other KIRs also exhibit distinguishable functional differences among their alleles (Steiner et al. 2008; Goodridge et al. 2007; Carr et al. 2005), although the exact molecular determinants have not been elucidated. Many human diseases are reported to be associated with differences in KIR gene content, including autoimmune diseases, inflammatory disorders, infectious diseases, immunodeficiency, cancer, and reproductive disorders (Kulkarni et al. 2008). The relation between these diseases and functional heterogeneity among the alleles of KIR is not yet known due to the lack of expedient methods for high throughput typing of different functional groups of KIR alleles.

KIRs recognize the highly polymorphic human leukocyte antigen (HLA) class 1 protein with unique specificity (Long et al. 1996; Parham, P. 2006). NK cell functions are inhibited when the inhibitory KIRs recognize their specific ligands on target cells. For example, KIR2DL1 and KIR2DL2/2DL3 recognize HLA-C allotypes whereas KIR3DL1 recognizes allotypes of HLA-B and -A. HLA-C ligands are divided into two groups (HLA-C1 and HLA-C2 respectively) based on the presence of asparagine or lysine at amino acid position 80 in the mature protein (Mandelboim et al. 1996). Furthermore, HLA-C1 contains a conserved serine residue at amino acid position 77, while an asparagine is present in HLA-C2 at the same position. KIR2DL1 recognizes HLA-C2 and KIR2DL2/2DL3 recognizes HLA-C1 (Colonna et al. 1993). HLA-B is also divided into two groups, HLA-Bw4 and HLA-Bw6, based on their differences in the amino acid position 77-83. HLA-B and HLA-A (A*23, A*24, A*32) alleles carrying the HLA-Bw4 epitopes are recognized by KIR3DL1 (Gumperz et al. 1995). HLA-Bw6 is not a ligand for KIRs. Disease susceptibility has been associated with various KIR ligand constellations.

Biologically, NK cell reactivity toward target cells is based in part on the presence of KIRs and their cognate ligands. Because not all alleles of a certain inhibitory KIR interact at the same degree with various HLA alleles of the same ligand group (De Santis et al. 2010), allelic polymorphism makes the prediction of eventual NK cell reactivity difficult. The precise prediction of NK activity requires the knowledge of the molecular determinants of both KIRs and their ligands. Thus, in some embodiments, the present invention contemplates a novel approach on how to develop a real time PCR based method to assess the different functional groups of KIR2DL1, built upon our knowledge of the unique molecular determinant. In addition, on other embodiments, the present invention further contemplates a parallel method for functional KIR-ligand typing. These methods provide rapid and cost effective information of biological and clinical significance.

### I. Discussion

Although numerous studies have established the importance of KIRs and their HLA ligand in health, disease, and transplant outcomes, the significance of allele polymorphism among these two sets of most highly polymorphic gene families has not yet been elucidated. The obstacles were two-fold: first, although it is certain that functional heterogeneity exists among different alleles, the molecular determinants are largely unknown; second, even if the molecular determinants are known, there is no expedient laboratory method for high volume testing. Herein, in some embodiments, the present invention contemplates a novel approach for rapid and cost effective typing of KIR alleles and KIR ligands once the molecular determinants are identified. Importantly, the results provide biologically informative data that will be useful for clinical diagnostics.

In the past, NK cell reactivity could be predicted in part based on the presence of KIRs and their ligands. However, allelic polymorphism generates another layer of functional heterogeneity. For the precise prediction of NK cell reactivity (e.g. in donor selection for NK cell transplantation), the knowledge of the presence of specific functional allelic group of KIRs and their ligands are important. For example only and not meant to be limiting, using the functional KIR allele group typing based on the knowledge of the molecular determinant, it was correctly predicted that KIR2DL1 R²⁴⁵/R²⁴⁵ NK cells were more reactive towards missing self when compared with KIR2DL1 C²⁴⁵/C²⁴⁵ cells. Thus, a clinical hypothesis might be that bone marrow transplant donors with KIR2DL1 R²⁴⁵/R²⁴⁵ are better than those with KIR2DL1 C²⁴⁵/C²⁴⁵ for a patient lacking HLA-C2 ligand. Another example, and not meant to be limiting, is pregnancy. When pregnant mothers who lack most or all activating KIR are carrying fetuses which possess KIR2DL1 ligand HLA-C2 group, they are at an increased risk of preeclampsia (Hiby et al. 2004). This is even true if mother herself also has HLA-C2. Since KIR2DL1 R²⁴⁵/R²⁴⁵ is more inhibitory than KIR2DL1 C²⁴⁵/C²⁴⁵, one may predict that if the mother possesses KIR2DL1 R²⁴⁵/R²⁴⁵, she may be at higher risk of developing preeclampsia when carrying a HLA-C2 positive baby. These mothers should then be monitored more closely during pregnancy.

Similar prediction is also possible for KIR2DL1 related diseases. For instance, it is believed that individuals who possess the KIR2DL1 R²⁴⁵/R²⁴⁵ alleles may have a decreased chance of developing autoimmune diseases when compared to individuals with the less inhibitory KIR2DL1 C²⁴⁵/C²⁴⁵ alleles. On the contrary, individuals carrying KIR2DL1 C²⁴⁵/C²⁴⁵ alleles may be at lower risk of cancer or chronic infection. With the high throughput KIR2DL1 functional group typing described herein, we should now be able to study these hypotheses expeditiously.

Similar to KIR alleles, current HLA-ligand typing required high resolution allele typing, which is expensive and time consuming. The SNP assay, presented herein, can type KIR-ligands for 94 individuals within 4 hours, and the cost is about 4 dollars per sample. On the contrary, high resolution HLA typing takes several weeks, and each sample costs about 200 dollars (Yun et al. 2007).

In summary, some embodiments of the present invention, contemplate a novel approach for KIR alleles and KIR-ligand typing that can be easily adopted in clinical diagnostic labs. Importantly, it is believed that the results are biologically informative in the prediction of NK cell activity. This information will be valuable for future biological study of NK cells in health and disease, and for clinical medicine in prognostication and transplant donor selection. This functional molecular-determinant based approach will also be useful for the development of rapid SNP assays for many other highly polymorphic loci in the entire human genome.

### II. Materials and Methods

### A. Cells, Culture, and Transduction

DNA samples used for KIR2DL1 alleles and KIR-ligands typing were obtained from PBMC of healthy donors at St. Jude Children's Research Hospital. The B-lymphoblastic cell line 721.221 was purchased from the International Histocompatibility Working Group and cultured in RPMI 1640 supplemented with 20% FBS and 1 mM penicillin/streptomycin. The 721.221 cells were transduced with retroviral vector MMP-IC-GFP-W containing HLA-Cw6 and HLA-Cw7. High-expressing cells were sorted by flow cytometric cell sorting using GFP expression. The K562 cell line was purchased from American Type Culture Collection (ATCC) and cultured in RPMI 1640 supplemented with 10% FBS and 1 mM penicillin/streptomycin.

### B. Detection of Effector NK Cell Reactivity Toward Target Cells

To separate the effector cells from target cell, effector cells were pre-labeled with pan-leukocyte marker CD45. The pre-labeled effector cells were washed extensively and then mixed with target cells. The CD107 cytotoxicity assay was performed as described by Bari et al. Briefly, pre-labeled effector PBMCs were mixed with target K562 or 721.221 cells and incubated at 37° C. in presence of anti-CD107a mAb antibody, clone H4A3 (BD Pharmingen). After 1 hour of coculturing, Golgi stop (BD Biosciences) was added to a final concentration 5 mM, followed by 3 hours of incubation in a cell culture incubator. Cells were then washed and labeled with an antibody cocktail containing appropriate antibodies (monoclonal antibody against KIR2DL1, KIR2DL2/2DL3, KIR3DL1 and NKG2a). Single KIR⁺ NK cell populations were gated from the CD45⁺ cell population, and cytotoxicity was measured based on CD107 surface expression by flow cytometry (LSR II cytometer). The absolute number of CD107⁺ cells in single KIR⁺ NK cell subsets was calculated using the following calculation:

$$\frac{No . of CD 107 mobilization in single {KIR}^{+} NK cell subset}{No . of lymphocytes in donor} \times {10}^{6} \text{/} ml$$

### C. Single Nucleotide Polymorphism (SNP) Assay

The single Nucleotide Polymorphism (SNP) assay was performed on the HT7900 from Applied Biosystems, following the allelic discrimination assay protocol provided by the manufacturer. Primers for the assay were designed in such a way that they amplified all the alleles of a particular HLA type (HLA-B or HLA-C) as well as the amplicon containing the polymorphic region of interest. Two probes were designed with a single mismatch between them. Each probe bound only one group of alleles and was labeled with either 6FAM or VIC fluorescent dye at their 5′ end. The probes also contained Taqman® minor groove binder (MGB) with non-fluorescent quencher (NFQ) (Applied Biosystems). For HLA-B typing, the universal primer pair that was designed to amplify all the alleles of HLA-B was: forward primer 5′-GAGGGGCCGGAGTATTGGGA-3′ (SEQ ID NO.: 1) and the reverse primer 5′-TGTAATCCTTGCCGTCGTAGG-3′ (SEQ ID NO.: 2). The probe for HLA-Bw4 associated HLA-B and -A was 6FAM-CCGCTACTACAACCAG-MGBNFQ (SEQ ID NO.: 3) and for HLA-Bw6 was VIC-CGGCTACTACAACCAG-MGBNFQ (SEQ ID NO.: 4). For HLA-C, forward primer 5′-TTGGGACCGGGAGACACAG-3′ (SEQ ID NO.: 5) and reverse primer 5′-CGATGTAATCCTTGCCGTC-3′ (SEQ ID NO.: 6) were used. The probes used for HLA-C1 and HLA-C2 was 6FAM-CCGAGTGAG CCTGC-MGBNFQ (SEQ ID NO.: 7) and VIC-CCGAGTGAA CCTGC-MGBNFQ (SEQ ID NO.: 8), respectively. Each assay reaction mix contained 250 nM probe concentration and 20 ng of genomic DNA in 1× Taqman genotyping master mix from Applied Biosystems, (USA). For KIR2DL1 functional allele typing, the probe was designed based on a single nucleotide mismatch at amino acid position 245 in mature protein. The sequences for the probes used to distinguish the two functional group of KIR2DL1 alleles are: 6FAM-CATCGCTGGTGCTC-MGBNFQ (SEQ ID NO.: 9), and VIC-CATTGCTGGTGCTCC-MGBNFQ (SEQ ID NO.: 10). Universal primer was designed that could specifically amplify all the alleles of KIR2DL1. The sequence of primer pair used was: forward primer 5′-CTCTTCATCCTCCTCTTCTTTC-3′ (SEQ ID NO.: 11) and reverse primer-5′-GAAAACGCAGTGATTCAACTG-3′ (SEQ ID NO.: 12). The SNP assay was run on the HT7900 from ABI using the same protocol as described for KIR ligand typing. The only exception was that the amount of DNA used to amplify the KIR2DL1 alleles was 50 ng instead of 20 ng.

### III. Results

### A. Molecular-Determinant Based KIR2DL1 Allele Typing.

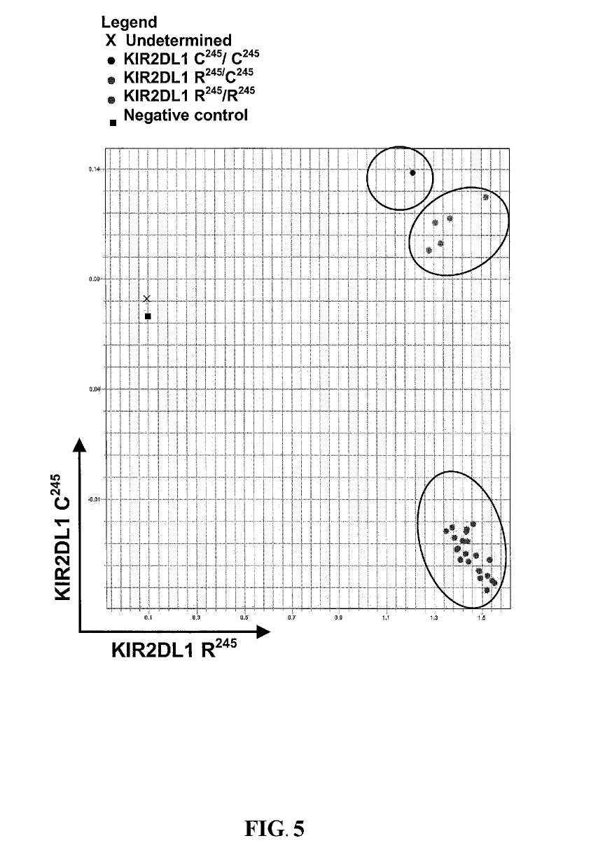

An arginine at amino acid position 245 of KIR2DL1 present in some alleles made them a stronger inhibitory receptor with more durable surface expression on NK cells, as compared to alleles that had a cysteine at the same position (Bari et al. 2009). In one embodiment, the present invention contemplates a SNP assay to type different functional groups of KIR2DL1 alleles. First, a universal primer pair to amplify all the alleles of KIR2DL1 was designed (SEQ ID NO.: 11 and 12, Materials and Methods). Next, two probes were designed in such a way that they contained a single nucleotide mismatch at position 796 (at amino acid position 245 in mature proteins) that distinguished the two functional groups of KIR2DL1 (SEQ ID NO.: 9 and 10, Materials and Methods). Although it is not necessary to understand the mechanism of an invention, the assay requires only a single nucleotide mismatch in the probe pair to discriminate two different allele groups. The SNP assay was evaluated using DNA from 27 donors (FIG. 5). Results showed that 20 individuals had only arginine at amino acid position 245 of KIR2DL1, 5 individuals were heterozygous for arginine and cysteine, and 1 individual had only cysteine in this position. All 26 PCR products were sequenced and confirmed the accuracy of the SNP assay. For one result, the individual designated as “undetermined,” there was no amplification of PCR products, indicating the absence of genomic KIR2DL1 (FIG. 5). The negative control result was similar to the result obtained for the undetermined result. Thus, the assay approach was not only useful for the identification of functional groups of KIR alleles, but also the presence of the KIR gene itself.

### B. Functional Relevance of Molecular-Determinant Based MR Typing.

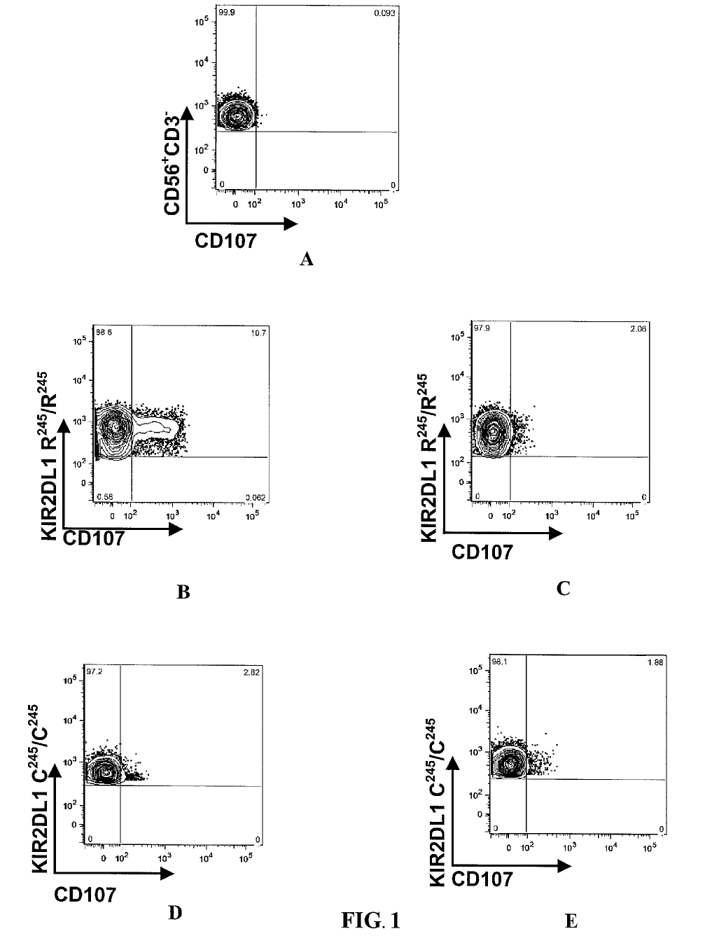

In one embodiment, the present invention contemplates validation of the accuracy of the KIR typing method in discerning NK cell activity. NK cells were isolated from donor PBMCs using CD56 microbeads by Automacs (Miltenyi). DNA was extracted from the same donor PBMCs and typed for the presence of KIR-ligands (HLA-C1 and HLA-C2) and different functional allelic groups of KIR2DL1 (KIR2DL1 R²⁴⁵ and KIR2DL1 C²⁴⁵). NK cells from donors with HLA-C2 were chosen and mixed with target cells 721.221 expressing HLA-Cw6 (HLA-C2) or HLA-Cw7 (HLA-C1). As expected, NK cells showed no CD107 expression on their surface in the absence of target cells (FIG. 1A). NK cells having KIR2DL1 R²⁴⁵ were more reactive against target cells expressing non-ligand Cw7 in comparison to KIR2DL1 C²⁴⁵ cells [7.5%±2.6% (average of six experiments) and 2.8%±1.1% (average of three experiments), respectively, p<0.05] (FIG. 1B, 1D). The reactivity against 721.221 in the presence of its ligand Cw6 were equally suppressed in KIR2DL1 R²⁴⁵ cells versus KIR2DL1 C²⁴⁵ cells (FIG. 1C, 1E). The above results confirm that the molecular-determinant based KIR typing method can accurately predict various degrees of NK cell activity against missing-self.

### C. SNP Assay to Detect MR Ligand Groups.

In one embodiment, the invention contemplates a method for ligand typing providing use for biological research and clinical applications. The current standard for KIR-ligand typing is high resolution HLA-typing, which is expensive and time consuming, so development of a rapid economical alternative would be of great interest.

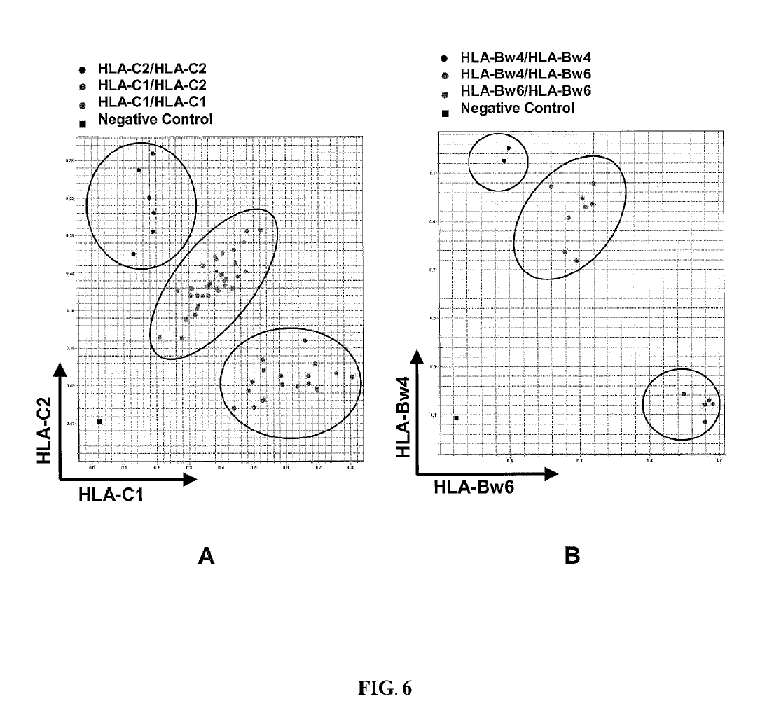

While it is not necessary to understand the mechanism of an invention, the ligands for KIR2DL1, KIR2DL2/2DL3, and KIR3DL1 are HLA-C2, HLA-C1, and HLA-Bw4, respectively. KIR3DL1 also recognizes some HLA-A alleles known as HLA-Bw4 associated HLA-A (A*23, A*24, A*32). HLA-Bw6 is not a ligand. KIR-ligand HLA-C1 contains a serine (S) at amino acid position 77, whereas HLA-C2 has an asparagine (N) at the same position. While it is not necessary to understand the mechanism of an invention, a probe pair, was designed, in such a way that it contained only one mismatch at nucleotide position 302 (amino acid position 77 in the mature protein) (SEQ ID NO.: 7 and 8, Materials and Methods). Further, a universal primer pair, that amplifies all the alleles of both HLA-C1 and HLA-C2, was designed (SEQ ID NO.: 5 and 6, Materials and Methods). Sixty (60) DNA samples were tested that were HLA typed at the allelic level for HLA-A, -B, and -C. The SNP assay was performed in a blinded study, e.g. run initially without prior knowledge of the donor HLA type. While it is not necessary to understand the mechanism of an invention, results demonstrated that the SNP assay can distinguish between HLA-C1 homozygous (19 samples fell in this category), HLA-C2 homozygous (6 samples fell in this category), as well as HLA-C1/HLA-C2 heterozygous conditions (35 samples fell in this category) (FIG. 6A). Unblinding of the HLA typing results, of these donor samples, revealed 100% agreement in KIR ligand assignment. To confirm the accuracy of SNP typing, an additional 47 individual donors were evaluated for HLA-C1 and HLA-C2 typing using both high resolution HLA typing as well as the KIR ligand SNP assay. Out of the 47 samples tested, 12 samples were HLA-C1, 23 samples were HLA-C1/HLA-C2 and 12 samples were HLA-C2 homozygous conditions. Results from both tests were again identical.

In one embodiment, the present invention contemplates a similar method for HLA-Bw4 and HLA-Bw6 typing. More particularly, the method contemplates distinguishing between different HLA-B groups. For example only, and not meant to be limiting, HLA-B consists of several hundreds of different alleles that contain two main groups based on KIR-ligands and non-ligands. The two-allele groups are HLA-Bw4 and HLA-Bw6. HLA-Bw4 alleles are ligands for KIR3DL1 while HLA-Bw6 are not KIR-ligands. An arginine at position 83 is conserved among all Bw4 alleles while a glycine, at position 83, is conserved for all Bw6 alleles. In addition, some alleles of HLA-A contain an arginine at position 83 and those alleles (HLA-A*23, A*24, and A*32) are also ligands for KIR3DL1. Thus, it is believed that based on the presence or absence of arginine or glycine, the invention contemplates an SNP assay that distinguishes between KIR-ligands and non-ligands by detecting the alleles (Bw4 and a few HLA-A) that are ligands for KIR3DL1.

While it is not necessary to understand the mechanism of the invention, the HLA-B type was aligned using the alignment tools in the IGMT/HLA database (http://www.ebi.ac.uk/imgt/hla/align.html) and a single base pair mismatch between HLA-Bw4 and HLA-Bw6 at nucleotide position 319 (amino acid position 83 in mature protein) was chosen to design the probe for the Bw4/Bw6 SNP assay (SEQ ID NO.: 3 and 4, Materials and Methods). In addition, a primer pair that amplified all the HLA-Bw4 and HLA-Bw6 alleles, was designed (SEQ ID NO.: 1 and 2, Materials and Methods). A comparison of the initial results, from the SNP assay and high resolution HLA typing, identified some discrepancies between these two tests when HLA-B only was considered, because HLA-Bw4 associated HLA-A alleles (A*23, A*24, A*32) has the same sequence in the probe area as HLA-Bw4 associated HLA-B alleles. In the SNP assay, donors who were negative for HLA-B associated Bw4 epitope but positive for HLA-Bw4 associated HLA-A were shown to be HLA-Bw4 positive. Since both HLA-Bw4 associated HLA-B and HLA-A are ligands for KIR3DL1, the SNP assay turns out to be ideal for rapidly detecting both HLA-B and -A associated KIR3DL1 ligands. First, DNA samples from 15 donors were tested for HLA-Bw4 and HLA-Bw6 typing using SNP. Out of the 15 samples tested, 2 samples were Bw4 homozygous, 8 samples were Bw4/Bw6 heterozygous and 5 samples were Bw6 homozygous (FIG. 6B). To confirm the accuracy of SNP typing, an additional 79 individual donors were evaluated for HLA-Bw4 and HLA-Bw6 typing using both high resolution HLA typing as well as KIR-ligand SNP assay. Out of a total of 94 samples tested for HLA-Bw4 and HLA-Bw6, 9 samples were HLA-Bw4 homozygous, 58 samples were HLA-Bw4/HLA-Bw6 heterozygous and 27 sample were HLA-Bw6 homozygous. Among the 94 samples, 93 SNP results matched with those of high resolution HLA typing, whereas one sample was HLA-Bw4 homozygous in the SNP assay, contrary to HLA-Bw4/HLA-Bw6 heterozygous in HLA typing. Further investigation of the HLA-Bw6 sample did not resolve the discrepancy. However, if the presence or absence of the clinically-relevant HLA-Bw4 ligand (since HLA-Bw6 is not a KIR ligand) was considered alone, none of the SNP assays gave false positive or negative results when compared to the HLA data. It is believed that the SNP assay can be reliably used for KIR-ligand typing.

### D. Functional Relevance of KIR-Ligand Group Typing.

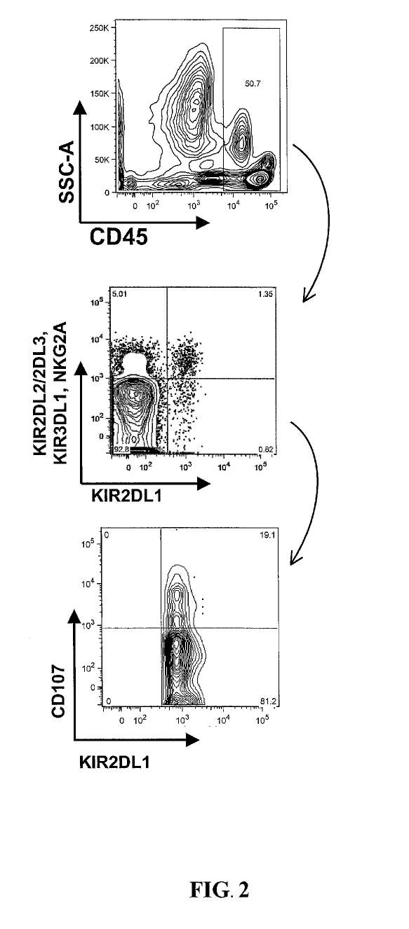

In one embodiment, the invention contemplates using the typing results to predict NK cell reactivity. Thus, effector PBMCs were pre-labeled with anti-CD45 mAb, to distinguish them from target K562 cells after mixing. K562 cells lack KIR-ligand expression. The gating strategies for the detection of CD 107 surface expression in KIR2DL1⁺ subsets of donor NK cells are shown in FIG. 2, where donor PBMCs were pre-labeled with CD45 and mixed with K562 cells. Single KIR+ NK cell subsets were gated using mAb against KIRs. The sequences of panels demonstrate the gating strategy of single KIR+ (first two panels) and degranulation of KIR2DL1+ (lowest panel) NK cell subset.

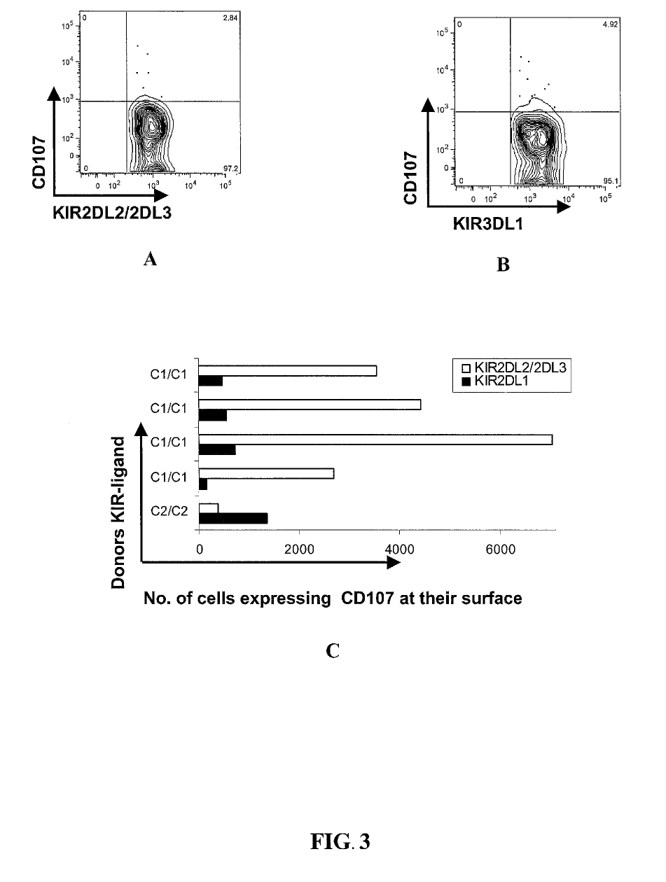

In this donor, who was positive for HLA-C2 but not for HLA-C1 and -Bw4, the single KIR2DL1⁺ subset showed the highest degranulation (19%) (FIG. 2), whereas KIR2DL2/KIR2DL3⁺ and KIR3DL1⁺ subsets showed much lower degranulation (FIG. 3A, 3B), indicating that NK cells could recognize specifically missing self. These results were then validated in 5 other donors (FIG. 3C), demonstrating the ability of our KIR-ligand assay in predicting NK cell activity through ligand participation in functional “licensing” or “education”.

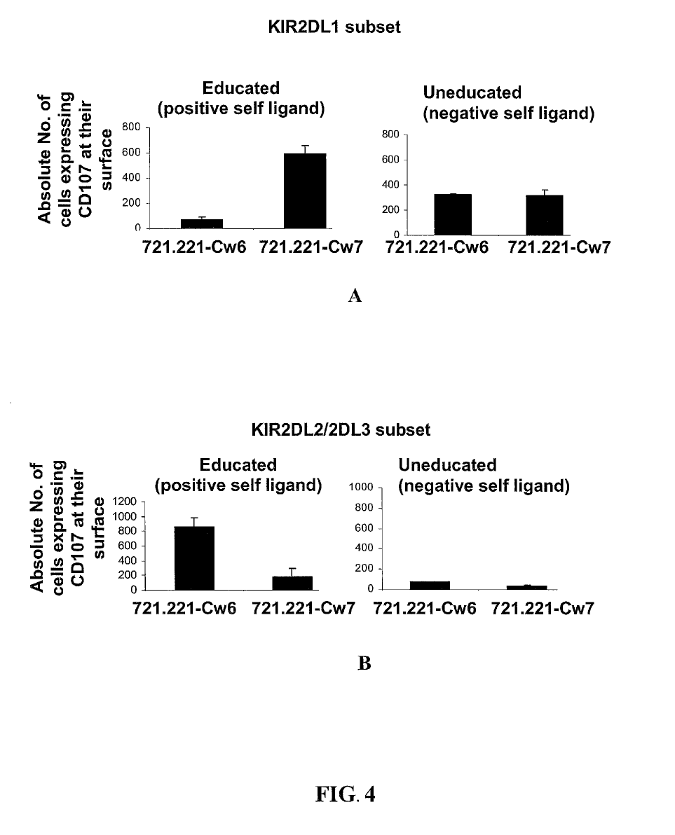

To further validate the accuracy of prediction, of NK cell licensing and recognition of missing self, based on our SNP results of the presence of a KIR ligand, we used 721.221 cells that ectopically expressed single KIR-ligand. Effector cells were pre-stained with CD45, gated on different single KIR⁺ NK cell subsets, and determined NK cell activation based on cell surface expression of CD107 as described in FIG. 2. Reactivity of educated versus uneducated KIR2DL1⁺ NK cell subsets based on the prediction of our SNP assay was highly predictable (FIG. 4A, 4B). Educated KIR2DL1⁺ NK cell subsets were able to discern the presence or absence of ligands, but uneducated cells were not (FIG. 4A). Similar results were also found in donor KIR2DL2/2DL3⁺ NK cell subsets (FIG. 4B). Therefore, it is believed that the developed SNP assay is a useful method for typing different functional groups of KIR ligands.

### EXPERIMENTAL

The following examples serve to illustrate certain preferred embodiments and aspects of the present invention and are not to be construed as limiting the scope thereof.

### Example I

### Molecular-Determinant Based KIR2DL1 Allele Typing

An arginine at amino acid position 245 of KIR2DL1 present in some alleles made them a stronger inhibitory receptor with more durable surface expression on NK cells, as compared to alleles that had a cysteine at the same position (Bari et al. 2009). In one embodiment, the present invention contemplates a SNP assay to type different functional groups of KIR2DL1 alleles. First, a universal primer pair to amplify all the alleles of KIR2DL1 was designed (SEQ ID NO.: 11 and 12, Materials and Methods). Next, two probes were designed in such a way that they contained a single nucleotide mismatch at position 796 (at amino acid position 245 in mature proteins) that distinguished the two functional groups of KIR2DL1 (SEQ ID NO.: 9 and 10, Materials and Methods). Although it is not necessary to understand the mechanism of an invention, the assay requires only a single nucleotide mismatch in the probe pair to discriminate two different allele groups. The SNP assay was evaluated using DNA from 27 donors (FIG. 5). Results showed that 20 individuals had only arginine at amino acid position 245 of KIR2DL1, 5 individuals were heterozygous for arginine and cysteine, and 1 individual had only cysteine in this position. All 26 PCR products were sequenced and confirmed the accuracy of the SNP assay. For one result, the individual designated as “undetermined,” there was no amplification of PCR products, indicating the absence of genomic KIR2DL1 (FIG. 5). The negative control result was similar to the result obtained for the undetermined result. Thus, the assay approach was not only useful for the identification of functional groups of KIR alleles, but also the presence of the KIR gene itself.

### Example II

### Functional Relevance of Molecular-Determinant Based MR Typing

In one embodiment, the present invention contemplates validation of the accuracy of the KIR typing method in discerning NK cell activity. NK cells were isolated from donor PBMCs using CD56 microbeads by Automacs (Miltenyi). DNA was extracted from the same donor PBMCs and typed for the presence of KIR-ligands (HLA-C1 and HLA-C2) and different functional allelic groups of KIR2DL1 (KIR2DL1 R²⁴⁵ and KIR2DL1 C²⁴⁵). NK cells from donors with HLA-C2 were chosen and mixed with target cells 721.221 expressing HLA-Cw6 (HLA-C2) or HLA-Cw7 (HLA-C1). As expected, NK cells showed no CD107 expression on their surface in the absence of target cells (FIG. 1A). NK cells having KIR2DL1 R²⁴⁵ were more reactive against target cells expressing non-ligand Cw7 in comparison to KIR2DL1 C²⁴⁵ cells [7.5%±2.6% (average of six experiments) and 2.8%±1.1% (average of three experiments), respectively, p<0.05] (FIG. 1B, 1D). The reactivity against 721.221 in the presence of its ligand Cw6 were equally suppressed in KIR2DL1 R²⁴⁵ cells versus KIR2DL1 C²⁴⁵ cells (FIG. 1C, 1E). The above results confirm that the molecular-determinant based KIR typing method can accurately predict various degrees of NK cell activity against missing-self.

### Example III

### SNP Assay to Detect MR Ligand Groups

In one embodiment, the invention contemplates a method for ligand typing providing use for biological research and clinical applications. The current standard for KIR-ligand typing is high resolution HLA-typing, which is expensive and time consuming, so development of a rapid economical alternative would be of great interest.

While it is not necessary to understand the mechanism of an invention, the ligands for KIR2DL1, KIR2DL2/2DL3, and KIR3DL1 are HLA-C2, HLA-C1, and HLA-Bw4, respectively. KIR3DL1 also recognizes some HLA-A alleles known as HLA-Bw4 associated HLA-A (A*23, A*24, A*32). HLA-Bw6 is not a ligand. KIR-ligand HLA-C1 contains a serine (S) at amino acid position 77, whereas HLA-C2 has an asparagine (N) at the same position. While it is not necessary to understand the mechanism of an invention, a probe pair, was designed, in such a way that it contained only one mismatch at nucleotide position 302 (amino acid position 77 in the mature protein) (SEQ ID NO.: 7 and 8, Materials and Methods). Further, a universal primer pair, that amplifies all the alleles of both HLA-C1 and HLA-C2, was designed (SEQ ID NO.: 5 and 6, Materials and Methods). Sixty (60) DNA samples were tested that were HLA typed at the allelic level for HLA-A, -B, and -C. The SNP assay was performed in a blinded study, e.g. run initially without prior knowledge of the donor HLA type. While it is not necessary to understand the mechanism of an invention, results demonstrated that the SNP assay can distinguish between HLA-C1 homozygous (19 samples fell in this category), HLA-C2 homozygous (6 samples fell in this category), as well as HLA-C1/HLA-C2 heterozygous conditions (35 samples fell in this category) (FIG. 6A). Unblinding of the HLA typing results, of these donor samples, revealed 100% agreement in KIR ligand assignment. To confirm the accuracy of SNP typing, an additional 47 individual donors were evaluated for HLA-C1 and HLA-C2 typing using both high resolution HLA typing as well as the KIR ligand SNP assay. Out of the 47 samples tested, 12 samples were HLA-C1, 23 samples were HLA-C1/HLA-C2 and 12 samples were HLA-C2 homozygous conditions. Results from both tests were again identical.

In one embodiment, the present invention contemplates a similar method for HLA-Bw4 and HLA-Bw6 typing. More particularly, the method contemplates distinguishing between different HLA-B groups. For example only, and not meant to be limiting, HLA-B consists of several hundreds of different alleles that contain two main groups based on KIR-ligands and non-ligands. The two-allele groups are HLA-Bw4 and HLA-Bw6. HLA-Bw4 alleles are ligands for KIR3DL1 while HLA-Bw6 are not KIR-ligands. An arginine at position 83 is conserved among all Bw4 alleles while a glycine, at position 83, is conserved for all Bw6 alleles. In addition, some alleles of HLA-A contain an arginine at position 83 and those alleles (HLA-A*23, A*24, and A*32) are also ligands for KIR3DL1. Thus, it is believed that based on the presence or absence of arginine or glycine, the invention contemplates an SNP assay that distinguishes between KIR-ligands and non-ligands by detecting the alleles (Bw4 and a few HLA-A) that are ligands for KIR3DL1.

While it is not necessary to understand the mechanism of the invention, the HLA-B type was aligned using the alignment tools in the IGMT/HLA database (http://www.ebi.ac.uk/imgt/hla/align.html) and a single base pair mismatch between HLA-Bw4 and HLA-Bw6 at nucleotide position 319 (amino acid position 83 in the mature protein) was chosen to design the probe for the Bw4/Bw6 SNP assay (SEQ ID NO.: 3 and 4, Materials and Methods). In addition, a primer pair that amplified all the HLA-Bw4 and HLA-Bw6 alleles, was designed (SEQ ID NO.: 1 and 2, Materials and Methods). A comparison of the initial results, from the SNP assay and high resolution HLA typing, identified some discrepancies between these two tests when HLA-B only was considered, because HLA-Bw4 associated HLA-A alleles (A*23, A*24, A*32) has the same sequence in the probe area as HLA-Bw4 associated HLA-B alleles. In the SNP assay, donors who were negative for HLA-B associated Bw4 epitope but positive for HLA-Bw4 associated HLA-A were shown to be HLA-Bw4 positive. Since both HLA-Bw4 associated HLA-B and HLA-A are ligands for KIR3DL1, the SNP assay turns out to be ideal for rapidly detecting both HLA-B and -A associated KIR3DL1 ligands. First, DNA samples from 15 donors were tested for HLA-Bw4 and HLA-Bw6 typing using SNP. Out of the 15 samples tested, 2 samples were Bw4 homozygous, 8 samples were Bw4/Bw6 heterozygous and 5 samples were Bw6 homozygous (FIG. 6B). To confirm the accuracy of SNP typing, an additional 79 individual donors were evaluated for HLA-Bw4 and HLA-Bw6 typing using both high resolution HLA typing as well as KIR-ligand SNP assay. Out of a total of 94 samples tested for HLA-Bw4 and HLA-Bw6, 9 samples were HLA-Bw4 homozygous, 58 samples were HLA-Bw4/HLA-Bw6 heterozygous and 27 sample were HLA-Bw6 homozygous. Among the 94 samples, 93 SNP results matched with those of high resolution HLA typing, whereas one sample was HLA-Bw4 homozygous in the SNP assay, contrary to HLA-Bw4/HLA-Bw6 heterozygous in HLA typing. Further investigation of the HLA-Bw6 sample did not resolve the discrepancy. However, if the presence or absence of the clinically-relevant HLA-Bw4 ligand (since HLA-Bw6 is not a KIR ligand) was considered alone, none of the SNP assays gave false positive or negative results when compared to the HLA data. These findings suggest that the SNP assay can be reliably used for KIR-ligand typing.

### Example IV

### Functional Relevance of KIR-Ligand Group Typing

In one embodiment, the invention contemplates using the typing results to predict NK cell reactivity. Thus, effector PBMCs were pre-labeled with anti-CD45 mAb, to distinguish them from target K562 cells after mixing. K562 cells lack KIR-ligand expression. The gating strategies for the detection of CD107 surface expression in KIR2DL1⁺ subsets of donor NK cells are shown in FIG. 2, where donor PBMCs were pre-labeled with CD45 and mixed with K562 cells. Single KIR+ NK cell subsets were gated using mAb against KIRs. The sequences of panels demonstrate the gating strategy of single KIR+ (first two panels) and degranulation of KIR2DL1+ (lowest panel) NK cell subset.

In this donor, who was positive for HLA-C2 but not for HLA-C1 and -Bw4, the single KIR2DL1⁺ subset showed the highest degranulation (19%) (FIG. 2), whereas KIR2DL2/KIR2DL3⁺ and KIR3DL1⁺ subsets showed much lower degranulation (FIG. 3A, 3B), indicating that NK cells could recognize specifically missing self. These results were then validated in 5 other donors (FIG. 3C), demonstrating the ability of our KIR-ligand assay in predicting NK cell activity through ligand participation in functional “licensing” or “education”.

To further validate the accuracy of prediction, of NK cell licensing and recognition of missing self, based on our SNP results of the presence of a KIR ligand, we used 721.221 cells that ectopically expressed single KIR-ligand. Effector cells were pre-stained with CD45, gated on different single KIR⁺ NK cell subsets, and determined NK cell activation based on cell surface expression of CD107 as described in FIG. 2. Reactivity of educated versus uneducated KIR2DL1⁺ NK cell subsets based on the prediction of our SNP assay was highly predictable (FIG. 4A, 4B). Educated KIR2DL1⁺ NK cell subsets were able to discern the presence or absence of ligands, but uneducated cells were not (FIG. 4A) Similar results were also found in donor KIR2DL2/2DL3⁺ NK cell subsets (FIG. 4B). Therefore, it is believed that the developed SNP assay is a useful method for typing different functional groups of KIR ligands.

### REFERENCES

All publications and patents mentioned in the above specification are herein incorporated by reference. Various modifications and variations of the described methods and devices of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention, which are obvious to those skilled in subject area such as biotechnology, molecular biology, immunology and-or related fields are intended to be within the scope of the following claims.
1. Lodoen M B, Lanier L L. Natural killer cells as an initial defense against pathogens. Curr Opin Immunol. 2006; 18:391-398.
2. Cerwenka A, Lanier L L. Natural killer cells, viruses and cancer. Nat Rev Immunol. 2001; 1:41-49.
3. Caligiuri M A. Human natural killer cells. Blood. 2008; 112:461-469.
4. Waldhauer I, Steinle A. NK cells and cancer immunosurveillance. Oncogene. 2008; 27:5932-5943.
5. Kim R, Emi M, Tanabe K. Cancer immunoediting from immune surveillance to immune escape. Immunology. 2007; 121:1-14.
6. Santoni A, Carlino C, Gismondi A. Uterine NK cell development, migration and function. Reprod Biomed Online. 2008; 16:202-210.
7. Moffett-King A. Natural killer cells and pregnancy. Nat Rev Immunol. 2002; 2:656-663.
8. Lanier L L. Up on the tightrope: natural killer cell activation and inhibition. Nat Immunol. 2008; 9:495-502.
9. Long E O, Colonna M, Lanier L L Inhibitory MHC class I receptors on NK and T cells: a standard nomenclature. Immunol Today. 1996; 17:100.
10. Parham P. The genetic and evolutionary balances in human NK cell receptor diversity. Semin Immunol. 2008; 20:311-316.
11. Robinson J, Marsh S G. IPD: the Immuno Polymorphism Database. Methods Mol Biol. 2007; 409:61-74.
12. Bari R, Bell T, Leung W H, et al. Significant functional heterogeneity among KIR2DL1 alleles and a pivotal role of arginine 245. Blood. 2009; 114:5182-5190.
13. Yawata M, Yawata N, Draghi M, Little A M, Partheniou F, Parham P. Roles for HLA and KIR polymorphisms in natural killer cell repertoire selection and modulation of effector function. J Exp Med. 2006; 203:633-645.
14. Steiner N K, Dakshanamurthy S, VandenBussche C S, Hurley C K. Extracellular domain alterations impact surface expression of stimulatory natural killer cell receptor KIR2DS5. Immunogenetics. 2008; 60:655-667.
15. Goodridge J P, Lathbury L J, Steiner N K, et al. Three common alleles of KIR2DL4 (CD158d) encode constitutively expressed, inducible and secreted receptors in NK cells. Eur J. Immunol. 2007; 37:199-211.
16. Can W H, Pando M J, Parham P. KIR3DL1 polymorphisms that affect NK cell inhibition by HLA-Bw4 ligand. J. Immunol. 2005; 175:5222-5229.
17. Kulkarni S, Martin M P, Carrington M. The Yin and Yang of HLA and KIR in human disease. Semin Immunol. 2008; 20:343-352.
18. Parham P. Taking license with natural killer cell maturation and repertoire development. Immunol Rev. 2006; 214:155-160.
19. Mandelboim O, Reyburn H T, Vales-Gomez M, et al. Protection from lysis by natural killer cells of group 1 and 2 specificity is mediated by residue 80 in human histocompatibility leukocyte antigen C alleles and also occurs with empty major histocompatibility complex molecules. J Exp Med. 1996; 184:913-922.
20. Colonna M, Brooks E G, Falco M, Ferrara G B, Strominger J L. Generation of allospecific natural killer cells by stimulation across a polymorphism of HLA-C. Science. 1993; 260:1121-1124.
21. Gumperz J E, Litwin V, Phillips J H, Lanier L L, Parham P. The Bw4 public epitope of HLA-B molecules confers reactivity with natural killer cell clones that express NKB 1, a putative HLA receptor. J Exp Med. 1995; 181:1133-1144.
22. De Santis D, Foley B A, John E, Senitzer D, Christiansen F T, Witt C S. Rapid, flow cytometric assay for NK alloreactivity reveals exceptions to rules governing alloreactivity. Biol Blood Marrow Transplant; 16:179-191 2010.
23. Hiby S E, Walker J J, O'Shaughnessy K M, et al. Combinations of maternal KIR and fetal HLA-C genes influence the risk of preeclampsia and reproductive success. J Exp Med. 2004; 200:957-965.
24. Yun G, Tolar J, Yerich A K, et al. A novel method for KIR-ligand typing by pyrosequencing to predict NK cell alloreactivity. Clin Immunol. 2007; 123:272-280.
25. Lanier, et al., NK Cell Recognition. Annu. Rev. Immunol. 2005; 23:25-74.
26. Uhrberg, Markus, The KIR Gene Family: Life in the Fast Lane of Evolution. Eur. J. Immunol. 2005; 35:10-15.
27. Vely et al., Regulation of Inhibitory and Activating Killer-Cell Ig-Like Receptor Expression Occurs in T Cells After Termination of TCR Rearrangements. J. Immunol. 2001; 166:2487-2494.

1. A probe comprising the nucleic acid sequence selected from the group consisting of a nucleic acid sequence of SEQ. ID. NO: 9, a nucleic acid sequence of SEQ. ID. NO: 10, a nucleic acid sequence with greater than 98 percent homology of SEQ. ID. NO: 9, a nucleic acid sequence with greater than 98 percent homology of SEQ. ID. NO: 10, a nucleic acid sequence with greater than 95 percent homology of SEQ. ID. NO: 9, a nucleic acid sequence with greater than 95 percent homology of SEQ. ID. NO: 10, a nucleic acid sequence with greater than 90 percent homology of SEQ. ID. NO: 9, a nucleic acid sequence with greater than 90 percent homology of SEQ. ID. NO: 10, wherein said probe is capable of distinguishing between the presence and absence of nucleic acid sequences coding for arginine and cysteine at position 245 of KIR2DL1.

2. A kit, comprising:
a) providing:
i. a probe from claim 1; and
ii. instructions for use.

3. A method, comprising:
a) providing:
i. a sample from a subject, wherein said sample comprises nucleic acid encoding KIRs.
ii. a plurality of primers wherein said primers can amplify all of the alleles of KIR2DL1;
iii. a plurality of probes wherein said probes can recognize the presence of nucleic acid sequences coding for arginine and cysteine at position 245; and
b) contacting said sample with said primers for a sufficient amount of time to amplify all of the alleles of KIR2DL1; and
c) determining the presence and absence of nucleic acid sequences coding for arginine and cysteine with said plurality of probes.

4. The method of claim 2, further comprising treating the subject with a first therapy depending on the presence of coding sequence for arginine.

5. The method of claim 2, further comprising treating the subject with a second therapy depending on the presence of coding sequence for cysteine.

6. A kit, comprising:
a) providing:
i. a plurality of primers and a plurality of probes for KIR typing wherein said typing includes distinguishing between the presence and absence of nucleic acid sequences coding for arginine and cysteine at position 245 of KIR2DL1; and
ii. instructions for use.

7. A probe comprising the nucleic acid sequence selected from the group consisting of a nucleic acid sequence of SEQ. ID. NO: 7, a nucleic acid sequence of SEQ. ID. NO: 8, a nucleic acid sequence with greater than 98 percent homology of SEQ. ID. NO: 7, a nucleic acid sequence with greater than 98 percent homology of SEQ. ID. NO: 8, a nucleic acid sequence with greater than 95 percent homology of SEQ. ID. NO: 7, a nucleic acid sequence with greater than 95 percent homology of SEQ. ID. NO: 8, a nucleic acid sequence with greater than 90 percent homology of SEQ. ID. NO: 7, a nucleic acid sequence with greater than 90 percent homology of SEQ. ID. NO: 8, wherein said probe is capable of distinguishing between the presence and absence of nucleic acid sequences coding for serine and asparagine at position 77 of HLA-C1/C2.

8. A kit, comprising:
a) providing:
i. a probe from claim 7; and
ii. instructions for use.

9. A method, comprising:
a) providing:
i. a sample from a subject, wherein said sample comprises nucleic acid encoding KIR ligands;
ii. a plurality of primers wherein said primers can amplify all of the alleles of HLA-C1/C2;
iii. a plurality of probes wherein said probes can recognize the presence of nucleic acid coding for a serine and asparagine at position 77 of HLA-C1/C2; and
b) contacting said sample with said primers for a sufficient amount of time to amplify all of the alleles of HLA-C1/C2; and
c) determining the presence and absence of nucleic acid sequences coding for serine and asparagine with said plurality of probes.

10. The method of claim 9, further comprising treating the subject with a first therapy depending on the presence of coding sequence for serine.

11. The method of claim 9, further comprising treating the subject with a second therapy depending on the presence of coding sequence for asparagine.

12. A kit, comprising:
a) providing:
i. a plurality of primers and a plurality of probes for KIR ligand typing wherein said typing includes distinguishing between the presence and absence of nucleic acid sequences coding for serine and asparagine at position 77 of HLA-C1/C2; and
ii. instructions for use.

13. A probe comprising the nucleic acid sequence selected from the group consisting of a nucleic acid sequence of SEQ. ID. NO: 3, a nucleic acid sequence of SEQ. ID. NO: 4, a nucleic acid sequence with greater than 98 percent homology of SEQ. ID. NO: 3, a nucleic acid sequence with greater than 98 percent homology of SEQ. ID. NO: 4, a nucleic acid sequence with greater than 95 percent homology of SEQ. ID. NO: 3, a nucleic acid sequence with greater than 95 percent homology of SEQ. ID. NO: 4, a nucleic acid sequence with greater than 90 percent homology of SEQ. ID. NO: 3, a nucleic acid sequence with greater than 90 percent homology of SEQ. ID. NO: 4, wherein said probe is capable of distinguishing between the presence and absence of nucleic acid sequences coding for arginine in HLA-Bw4 and HLA-A, and glycine in HLA-Bw6 at position 83.

14. A kit, comprising:
a) providing:
i. a probe from claim 13; and
ii. instructions for use.

15. A method, comprising:
a) providing:
i. a sample from a subject, wherein said sample comprises nucleic acid encoding KIR ligands HLA-B and -A;
ii. a plurality of primers wherein said primers can amplify all of the alleles of HLA-B/A;
iii. a plurality of probes wherein said probes can recognize the presence of nucleic acid coding for a arginine in HLA-Bw4 and HLA-A and a glycine in HLA-Bw6 at position 83; and
b) contacting said sample with said primers for a sufficient amount of time to amplify all of the alleles of HLA-B/A; and
c) determining the presence and absence of nucleic acid sequences coding for arginine and glycine with said plurality of probes.

16. The method of claim 15, further comprising treating the subject with a first therapy depending on the presence of coding sequence for arginine.

17. The method of claim 15, further comprising treating the subject with a second therapy depending on the presence of coding sequence for glycine.

18. A kit, comprising:
a) providing:
i. a plurality of primers and a plurality of probes for KIR ligands typing wherein said typing includes distinguishing between the presence and absence of nucleic acid sequences coding for arginine in HLA-Bw4 and HLA-A, and glycine in HLA-Bw6 at position 83; and
ii. instructions for use.
